(19) United States
(12) Patent Application Publication
(10) Pub. No.: US 2002/0058948 A1
(43) Pub. Date: May 16, 2002

(54) Targeting system and method for distal locking of intramedullary nails

(76) Inventor: Yvan Arlettaz, Monthey (CH)

Correspondence Address: Ivor R. Elrifi, MINTZ LEVIN COHN FERRIS GLOVSKY AND POPEO PC, Suite 400, 11911 Freedom Drive, Reston, VA, 20190, US

(21) Appl. No.: 09/973,696

(22) Filed: Oct. 11, 2001

Related U.S. Application Data
(60) Provisional application No. 60/239,789, filed on Oct. 12, 2000.

(51) Int. Cl. A61B 17/58

(52) U.S. Cl. 606/98

(57) ABSTRACT

A targeting system and method is disclosed for the distal locking of intramedullary nails. The targeting system comprises a first fixation system, a second fixation system, and an aiming guide. After implantation within the intramedullary canal of a bone, the distal screw holes of an intramedullary nail are first aligned using conventional radioscopy equipment. The targeting system is then introduced between the radioscopy equipment and the patient such that the aiming guide of the targeting system is roughly aligned with the distal screw holes. The first fixation system is adapted to be fixedly coupled to an operating surface upon which an intramedullary nail fixation procedure may be performed. The second fixation system is adpated to be fixedly coupled to the handle of the implanted intramedullary nail. Rough adjustments to the first and second fixation systems enable an initial positioning of a drill with a first distal screw hole. The aiming guide, which is disposed between and fixedly coupled to the first fixation system and the second fixation system, is used to perfect alignment of the drill with the first distal screw hole. Once a precise alignment with the first distal screw hole has been realized, a mechanical adjustment to the aiming guide may allow for a rapid alignment of the drill with the second distal screw hole of the implanted intramedullary nail.

### CROSS-REFERENCE TO RELATED APPLICATIONS

This Application claims priority from U.S. Provisional Patent Application SerialNo. 60/239,789 filed Oct. 12, 2001, which is incorporated herein by reference.

### FIELD OF THE INVENTION

This invention relates generally to the field of surgery, and more particularly to a targeting system and method for the distal locking of intramedullary nails.

### BACKGROUND OF THE INVENTION

Known methods for treating fractures of the tibia, femur, humerus and other long bones of the body tend to focus on reducing, aligning, and stabilizing the fractured bone. In one known method, the intramedullary canal of each fractured bone segment may be reamed to allow for the passage of a tubular metal rod, known as an intramedullary nail. An intramedullary nail is typically inserted into the intramedullary canal of a fractured bone at a proximal or distal segment of the bone, and advanced through the intramedullary canal to a distal or proximal segment of the bone. To securely position an intramedullary nail within a bone, one or more holes may first be drilled through the wall of the bone. These holes are generally transverse to the major axis of the bone, and are in direct alignment with one or more screw holes pre-formed in proximal and distal portions of the intramedullary nail. Locking fasteners may then be inserted directly through the holes in the wall of the bone into the screw holes in the intramedullary nail. By locking portions of fractured bone segments to an implanted intramedullary nail, rotation, angulation, or shortening of the bone about the implanted intramedullary nail may be prevented.

Many specialists have acknowledged the benefits of intramedullary nailing fixation over other fixation techniques such as external fixation, and fixation by plate and screw. If successful, intramedullary nailing fixation may provide for the precise reduction and immediate stability of fractured bones. In addition, intramedullary nailing fixation may further ensure that proper axial alignment is maintained throughout the duration of the healing process.

Despite acknowledging these and other advantages, many specialists are reluctant to perform intramedullary nailing fixation procedures because of the difficulty associated with locating the screw holes in the distal portion of the insertion point of an intramedullary nail after the nail has been implanted in the intramedullary canal of a bone. After implantation, the precise locations of screw holes in the distal portion of the intramedullary nail may have to be identified so that distal locking fasteners can be drilled through the wall of the bone and placed directly in the distal screw holes of the intramedullary nail. These distal locking fasteners secure the distal portion of the intramedullary nail to the bone. As an intramedullary nail is being advanced through the intramedullary canal of a bone, however, it may be susceptible to pressure in flexion and torsion. The magnitude of this pressure may result in the bending and rotation of the intramedullary nail, and may fluctuate from patient to patient based on varying anatomical bone shape. Accordingly, it is not unusual for the position of the screw holes in the distal portion of the intramedullary nail to have deviated from their expected position as a result of implantation. As a result, if a drill is not precisely aligned with the screw holes in the distal portion of the intramedullary nail while drilling through the wall of the bone, the drill may miss the distal locking holes of the intramedullary nail, resulting in an improper stabilization of the fracture, and therefore in a potential failure of the treatment. Additionally, a fractured bone may be further weakened by holes that have to be re-drilled because of an initial misalignment.

The positioning of proximal locking fasteners for locking the proximal portion of an intramedullary nail to a bone, by contrast, is generally not as complicated. This is because screw holes in the proximal portion of an intramedullary nail may be located relatively close to the site of insertion in the bone, where their position may be easily predicted.

A number of different techniques have been used to locate distal screw holes in an implanted intramedullary nail for alignment with a drill. Many of these techniques, however, rely on the use of conventional X-ray equipment. This may be disadvantageous as X-ray images are often displayed on remote screens or monitors, which may force a specialist to continually look away from the patient to verify proper alignment. Moreover, prolonged exposure to radiation may negatively impact both the health of the patient and those performing the procedure.

In an effort to reduce exposure to X-ray radiation, some techniques have combined the use of X-ray equipment with a laser light source. An X-ray machine may first be used to locate a distal screw hole in an implanted intramedullary nail. A laser light spot may then be used to mark the location on the bone where the drill should be placed, allowing the X-ray machine to be turned off. One drawback associated with this technique, however, is that the light spot marking the position of the screw hole may be obstructed by both the drill and the hand of the surgeon operating the drill, which may affect alignment.

Additional techniques for locating distal screw holes in intramedullary nails utilize radioscopic equipment coupled to hand-guided instruments. Procedures performed according to this technique may be rigorous for a surgeon because of the level of dexterity and precision required to correctly perform the procedure within an acceptable operating time.

Still other techniques for locating distal screw holes in implanted intramedullary nails range from the use of magnets to the use of various elaborate sensor systems. Many of these systems may be quite expensive, however, and tend to require regular maintenance. These and other drawbacks exist.

### SUMMARY OF THE INVENTION

The invention solving these and other problems relates to a targeting system and method for the distal locking of intramedullary nails.

According to an embodiment of the invention, a targeting system comprising a first fixation system, a second fixation system, and an aiming guide is provided to facilitate the complex task of locating distal screw holes in an implanted intramedullary nail. After implantation within the intramedullary canal of a bone, the distal screw holes of an intramedullary nail may first be aligned using conventional radioscopy equipment. The targeting system may then be introduced between the radioscopy equipment and the patient such that the aiming guide of the targeting system is roughly aligned with the distal screw holes.

The first fixation system may be fixedly coupled to a surface that is integral with an operating table, or to any other surface upon which an intramedullary nail fixation procedure may occur. The second fixation system may be fixedly coupled to the handle of the implanted intramedullary nail. The aiming guide may be disposed between and fixedly coupled to the first fixation system and the second fixation system. As a result, the targeting system may be linked to an operating surface at a first end, and to a patient at a second end, while still remaining situated between the patient and the radioscopy equipment. Rough adjustments to the first and second fixation systems may enable an initial positioning of a drill with a first distal screw hole of the implanted intramedullary nail. The aiming guide of the targeting system may then be used to perfect alignment of the drill with the first distal screw hole. Once a precise alignment with the first distal screw hole has been realized and the first distal locking fastener fixed, a minimal mechanical adjustment to the aiming guide may allow for a rapid alignment of the drill with the second distal screw hole of the implanted intramedullary nail.

One advantage of the invention is that the targeting system may be linked to an operating surface at a first end, and to a patient at a second end. Since the targeting system is not directly coupled to any radioscopic equipment, adjustments to the targeting system may be made independent of any radioscopy equipment. In addition, the ability to adjust the targeting system out of the x-ray field may result in the reduction of a surgeon's exposure to irradiation during a procedure.

Another advantage of the invention is that the targeting system may be used with most conventional radioscopy equipment.

Yet another advantage of the invention is that radioscopy equipment may only be needed to assist in the alignment of a first distal screw hole in an implanted intramedullary nail. Once alignment with the first distal screw hole has been realized and the first distal locking fastener fixed, a minimal mechanical adjustment to the aiming guide of the targeting system may allow for a rapid alignment of the drill with the second distal screw hole of the implanted intramedullary nail. As such, both a patient and those performing the procedure may experience a reduction in exposure to irradiation during the procedure. In addition, an overall reduction in the duration of the procedure may also be experienced.

Still yet another advantage of the invention is that the targeting system may be used with almost any conventional or commercially available intramedullary nail and handle.

Still yet another advantage of the invention is that the targeting system is largely a visual system, making it an easy to learn, simple, and repeatable operation technique.

Another advantage of the invention is that the targeting system is a fully mechanical system, which may be less expensive and easier to maintain than other alignment systems that rely on magnets, or which utilize elaborate sensor technology.

These and other objects, features, and advantages of the invention will be apparent through the detailed description of the preferred embodiments and the drawings attached hereto. It is also to be understood that both the foregoing general description and the following detailed description are exemplary and not restrictive of the scope of the invention.

### BRIEF DESCRIPTION OF THE DRAWINGS

FIG. 1 is an illustrative example of an intramedullary nail implanted in the intramedullary canal of a long bone having a simple fracture.

FIG. 2 is an illustration of a targeting system, according to an embodiment of the invention.

FIG. 3 is an illustration of a targeting system, according to an embodiment of the invention.

FIG. 4 illustrates a first fixation system, according to an embodiment of the invention.

FIG. 5 is an illustration of a second fixation system, according to an embodiment of the invention.

FIG. 6 illustrates a second fixation system, according to an embodiment of the invention.

FIG. 7 is a rear view of an aiming guide, according to an embodiment of the invention.

FIG. 8 is a front view of an aiming guide, according to an embodiment of the invention.

FIGS. 9A-9C illustrate maximal displacement positions of an aiming guide, according to an embodiment of the invention.

FIG. 10 is an exploded view of an internal ring of an aiming guide, according to an embodiment of the invention.

FIGS. 11A-11B illustrate frontal views of the respective central positions and maximal eccentric positions of an aiming guide, according to an embodiment of the invention.

FIGS. 12A-12B illustrate a correct concentric alignment and an improper alignment, respectively, of an aiming guide with screw holes in a distal portion of an implanted intramedullary nail, according to an embodiment of the invention.

### DETAILED DESCRIPTION OF PREFERRED EMBODIMENTS

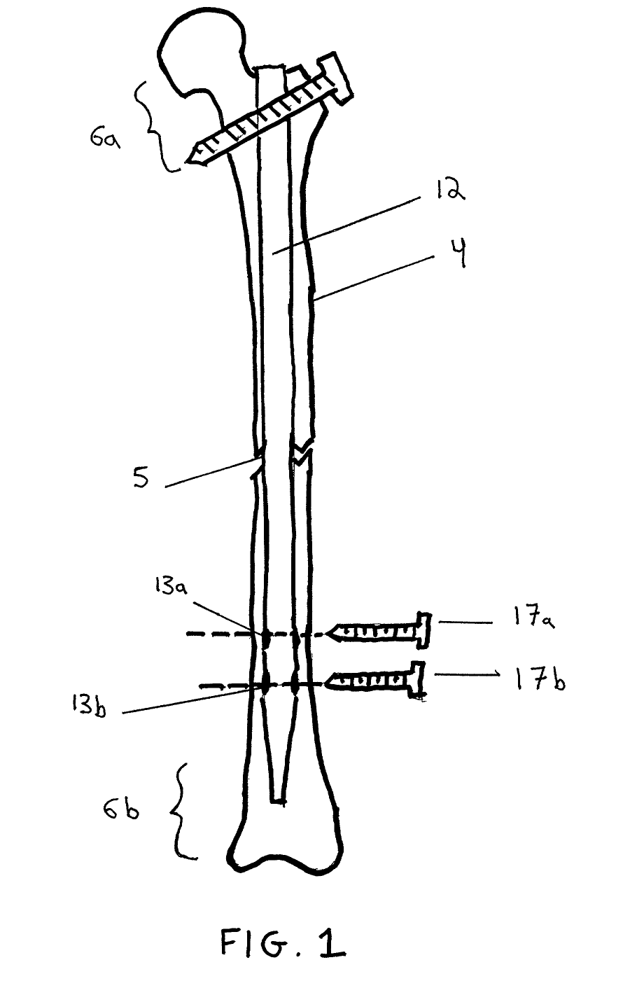

FIG. 1 is an exemplary illustration of an intramedullary nail 12 that has been inserted into the intramedullary canal of a bone 4 at a proximal bone segment 6a and advanced through the canal to a distal bone segment 6b. After implantation, the precise position of screw holes (13a, 13b) in the distal portion of implanted intramedullary nail 12 should be determined so that distal locking fasteners (17a, 17b) can be drilled through the wall of bone 4 and inserted directly into screw holes (13a, 13b). When properly secured within bone 4, intramedullary nail 12 may enable the repair and healing of a fracture 5 by stabilizing and maintaining a proper axial alignment of bone segments 6a and 6b.

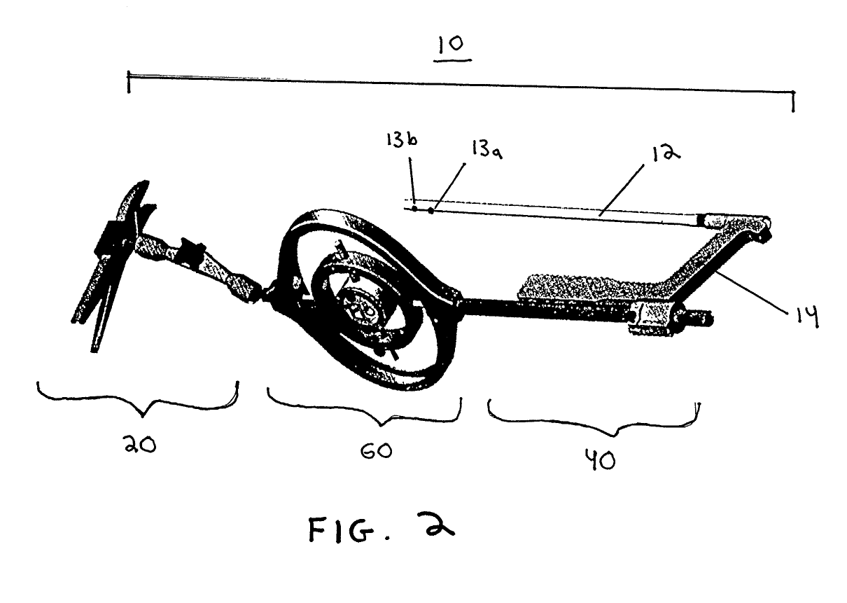

According to an embodiment of the invention illustrated in FIG. 2, a targeting system 10 is provided to facilitate the complex task of locating distal screw holes (13a, 13b) in implanted intramedullary nail 12. Targeting system 10 may comprise a first fixation system 20, a second fixation system 40, and an aiming guide 60. Once intramedullary nail 12 has been implanted within the intramedullary canal of bone 4, conventional radioscopy equipment (not shown) may first be aligned with distal screw holes (13a, 13b). Targeting system 10 may then be introduced between the radioscopy equipment and the patient such that an internal ring of aiming guide 60, discussed in greater detail below, is roughly aligned with distal screw holes (13a, 13b). At this point, first fixation system 20 may be fixedly coupled to an operating table or other operating surface, and second fixation system 40 may be fixedly coupled to the handle 14 of intramedullary nail 12. As a result, targeting system 10 may be linked to an operating surface at a first end, and to a patient at a second end, while still remaining situated between the patient and the radioscopy equipment. It should be recognized that first fixation system 20 and second fixation system 40 may be coupled to the operating surface and handle, respectively, in any order.

Rough adjustments to first fixation system 20 and second fixation system 40 may enable an initial positioning of a drill with a first distal screw hole, such as for example, distal screw hole 13a. Aiming guide 60 may then be used to perfect alignment of a drill with distal screw hole 13a. Once a precise alignment and fixation with distal screw hole 13a has been realized, a minimal mechanical adjustment to aiming guide 60 may allow for a rapid alignment of a drill with the second distal hole 13b. Having provided a general overview, the system and method of the invention will now be discussed in detail.

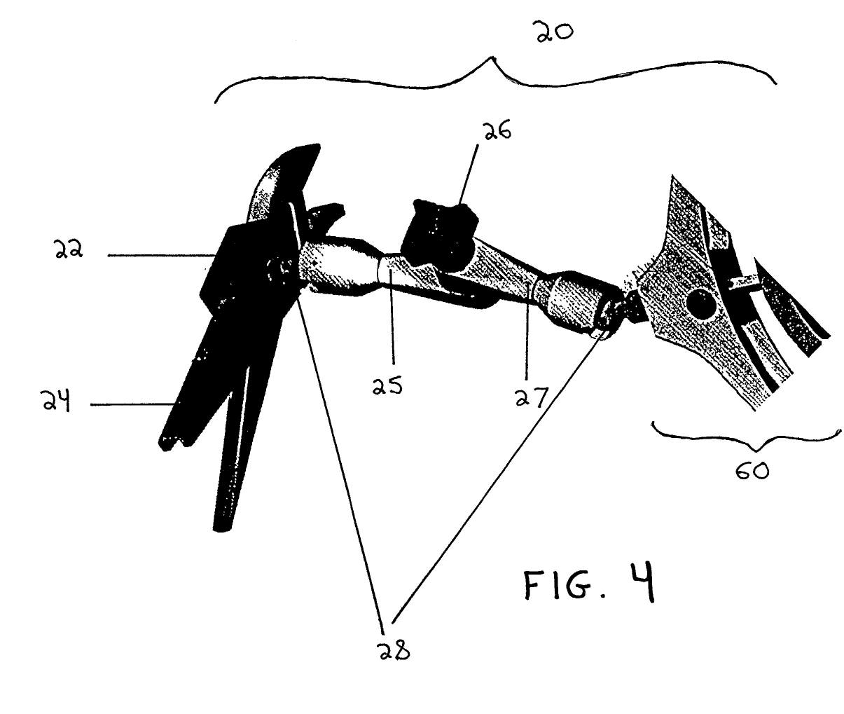

FIG. 4 illustrates a detailed view of first fixation system 20. To assist in positioning targeting system 10 between the radioscopy equipment and the patient, first fixation system 20 may be fixedly coupled to an operating table or other operating surface. According to one embodiment, first fixation system 20 comprises a first arm 25 that may releasably engage a second arm 27, via an adjustment mechanism 26. Adjustment mechanism 26 may comprise, for example, a series of inter-meshing gears or teeth. It should be recognized, however, that any mechanism enabling first arm 25 and second arm 27 to rotate with respect to one another may also be used.

At an opposite end from the end secured to second arm 27, first arm 25 may be linked to a clamp support 22 via a connection system 28. Connection system 28 may, for example, comprise a ball and socket joint, or any other suitable connection allowing for a 360 degree rotation of clamp support 22. Clamp support 22 may house a clamp 24 which can be secured to a surface that is integral with an operating table, or to any surface upon which an intramedullary nail fixation procedure may occur.

Additionally, as depicted in FIG. 4, second arm 27 may be linked to aiming guide 60 (at an opposite end from the end secured to first arm 25) via a similar connection system 28. Each of the aforementioned components of first fixation system 20 may be fabricated from any suitable surgical grade, bio-compatible materials, such as, for example, stainless steel, ceramics, titanium, or plastics. Other materials may be used.

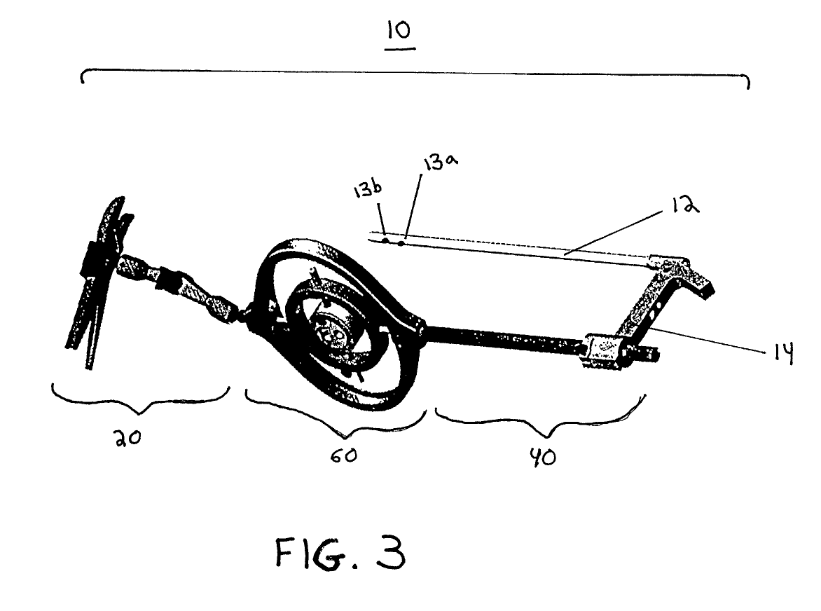

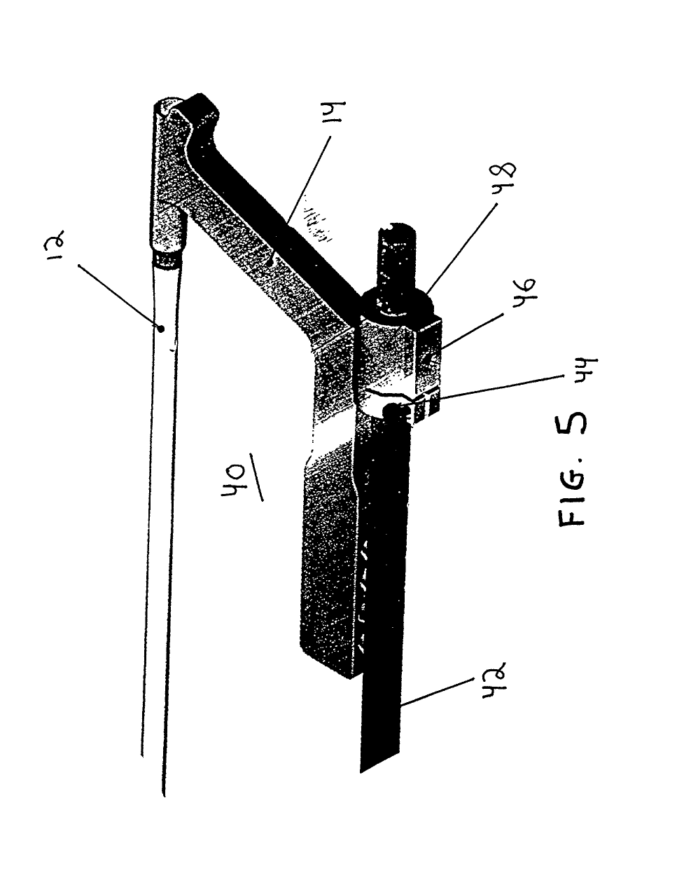

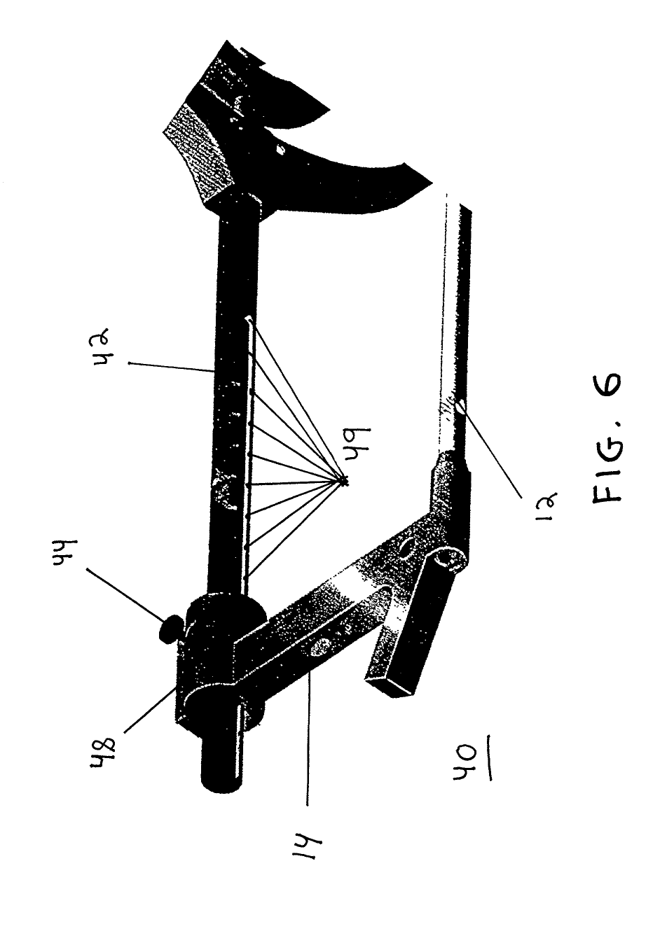

After first fixation system 20 has been fixedly coupled to a suitable operating surface, second fixation system 40 may be fixedly coupled to the handle 14 of intramedullary nail 12. Those having skill in the art will recognize that second fixation system 40 may be adapted for used with any conventional or commercially available intramedullary nail 12 and handle 14. In FIGS. 2 and 5, for example, nail 12 and handle 14 are depicted as a Gamma nail and handle, while in FIGS. 3 and 6, nail 12 and handle 14 are illustrated as a GK nail and handle.

With reference to FIGS. 5-6, second fixation system 40 may comprise a tubular clamp 48 that may either be integral with (or fixedly coupled to) handle 14. A translation rod 42 is also provided that may slidingly engage tubular clamp 48 at one end, and be integral with (or fixedly coupled to) aiming guide 60 at an opposite end.

Translation rod 42 may further comprise a plurality of notches 49 or detents at spaced intervals along its length that may correspond to scaled measurements of the length of nail 12. If, for example, the distance between the base of intramedullary nail 12 and the center of distal screw holes (13a, 13b) is known, translation rod 42 may be positioned according to which of notches 49 most accurately represents this distance. Tubular clamp 48 may also house a pressure spring 46 that is biased to engage any of notches 49 to secure translation rod 42 in a desired position along its path of translational movement. A locking screw 44 may also be provided to control the rotation of translation rod 42 within tubular clamp 48. The aforementioned components of second fixation system 40 may also be fabricated using any suitable surgical grade, bio-compatible materials, such as, for example, stainless steel, ceramics, titanium, or plastics. Other materials may be used.

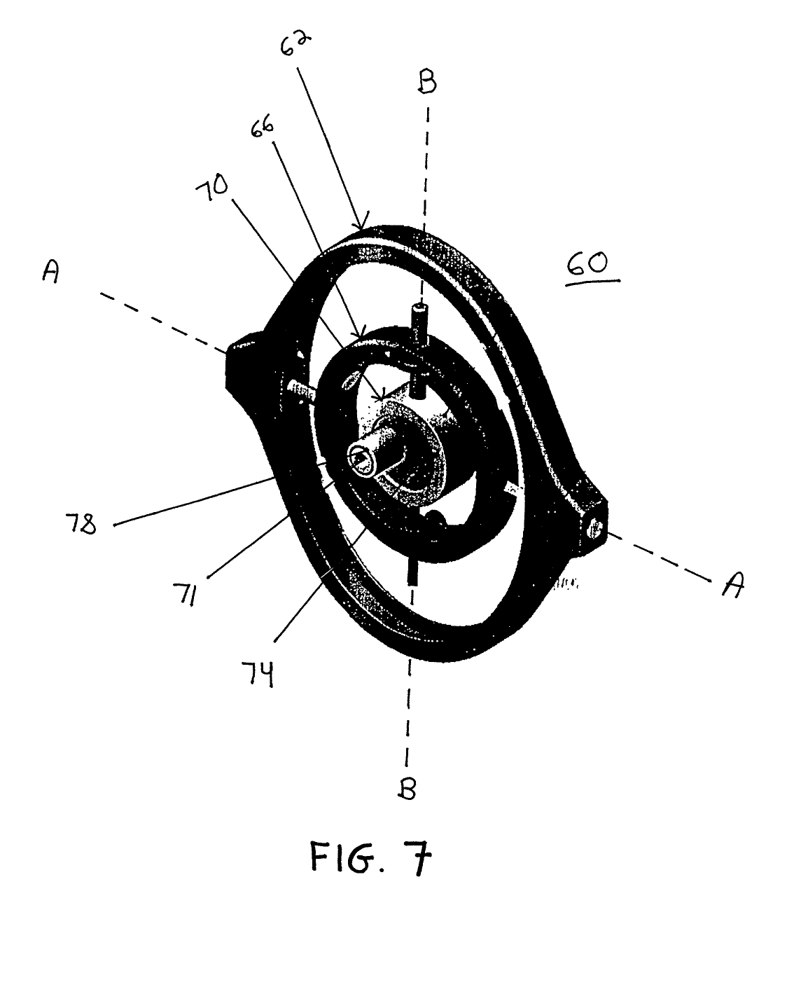

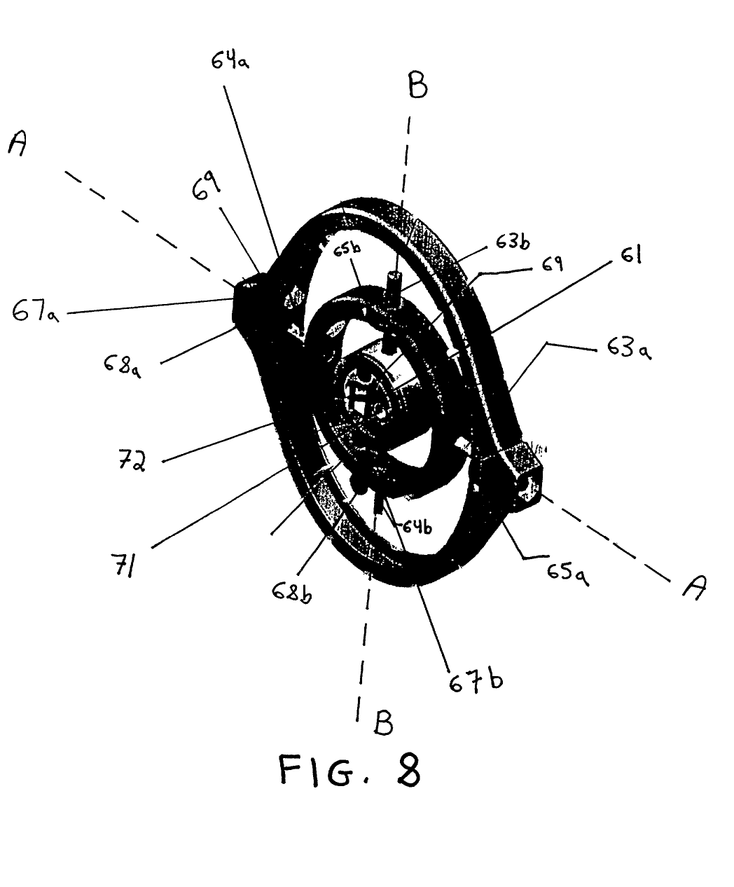

As previously described, adjustments to first fixation system 20 and second fixation system 40 may enable a rough, initial positioning of a drill with distal screw holes (13a, 13b). Aiming guide 60 may then be used to perfect alignment of a drill with a first distal hole, such as, for example, distal hole 13a. As illustrated in FIGS. 7-8, aiming guide 60 may be comprised of an external ring 62, an intermediate ring 66, and an internal ring 70, each of which may be fabricated from any suitable surgical grade, bio-compatible materials, such as, for example, stainless steel, ceramics, titanium, or plastics. Other materials may be used.

External ring 62 may enable intermediate ring 66 to move in translation along a central horizontal axis of external ring 62 (axis A), and in rotation about the central horizontal axis of external ring 62 (axis A). To effectuate movement in translation along axis A, a translation adjustment screw 63a mounted to intermediate ring 66 may be received by a translation adjustment button 65a that is integral with external ring 62. By rotating translation adjustment button 65a in either a clockwise or counter-clockwise direction, translation adjustment screw 63a may slide back and forth through translation adjustment button 65a, and along axis A. A bearing 67a allows for the rotation of a translation adjustment square 64a that is mounted to intermediate ring 66. Bearing 67a may be fixed by a fixing screw 68a to control rotation. Even if bearing 67a is fixed by fixing screw 68a, however, translation adjustment square 64a may still slide in translation along axis A.

Intermediate ring 66 may enable internal ring 70 to move in translation along a central vertical axis of intermediate ring 66 (axis B), and in rotation about the central vertical axis of intermediate ring 66 (axis B). As shown, internal ring 70 may move in a plane situated at ninety degrees compared to the displacement of the intermediate ring 66 with respect to the external ring 62. To effectuate movement in translation along axis B, a translation adjustment screw 63b mounted to internal ring 70 may be received by a translation adjustment button 65b that is integral with intermediate ring 66. By rotating translation adjustment button 65b in either a clockwise or counter-clockwise direction, translation adjustment screw 63b may slide back and forth through translation adjustment button 65b, and along axis B. A bearing 67b allows for the rotation of a translation adjustment square 64b that is mounted to internal ring 70. Bearing 67b may be fixed by a fixing screw 68b to control rotation. Even if bearing 67b is fixed by fixing screw 68b, however, translation adjustment square 64b may still slide in translation along axis B.

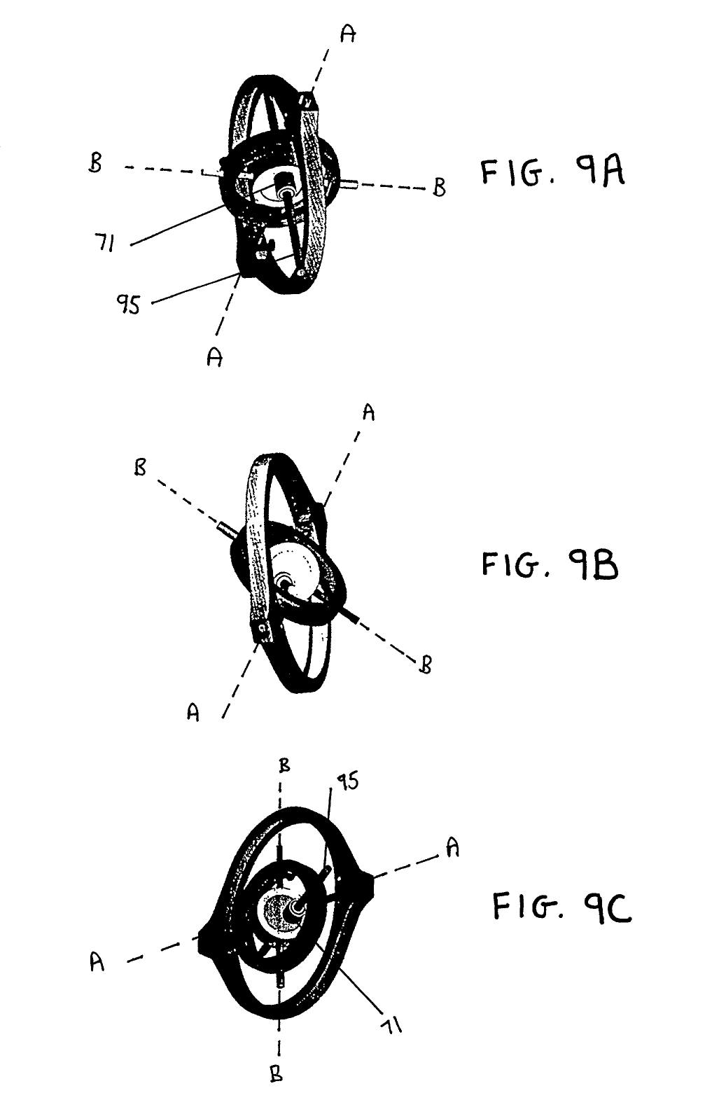

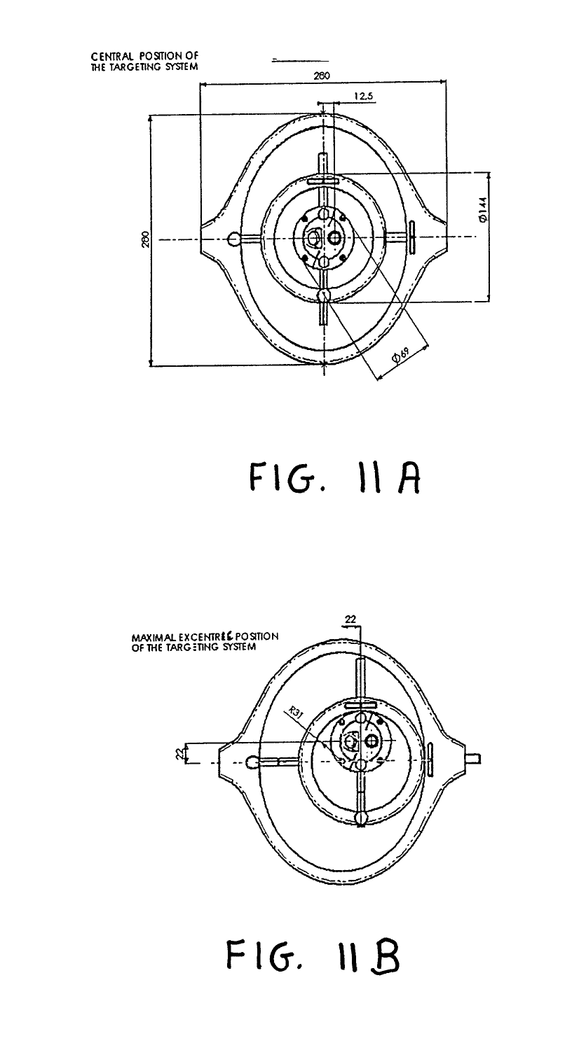

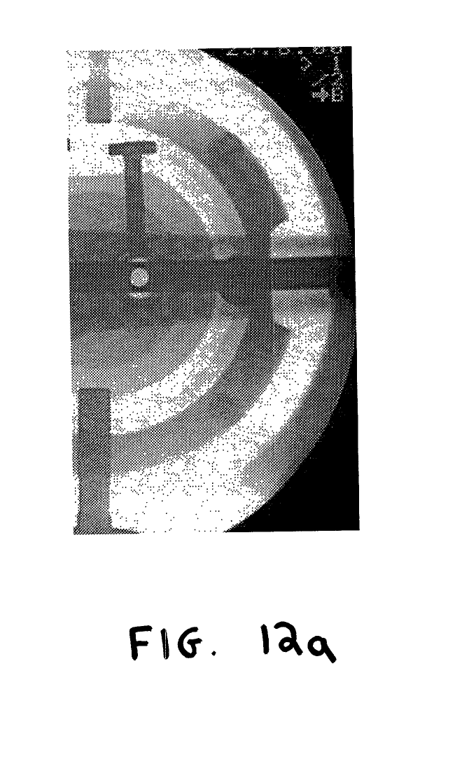

FIGS. 9A-9C are illustrations of maximal displacement positions of intermediate ring 66 about axis A, and of internal ring 70 about axis B. For exemplary purposes, these figures depict a drilling guide 95 secured within an aiming cylinder 71. FIGS. 11A-11B illustrate a frontal view of the respective central positions and maximal eccentric positions of aiming guide 60, according to an embodiment of the invention.

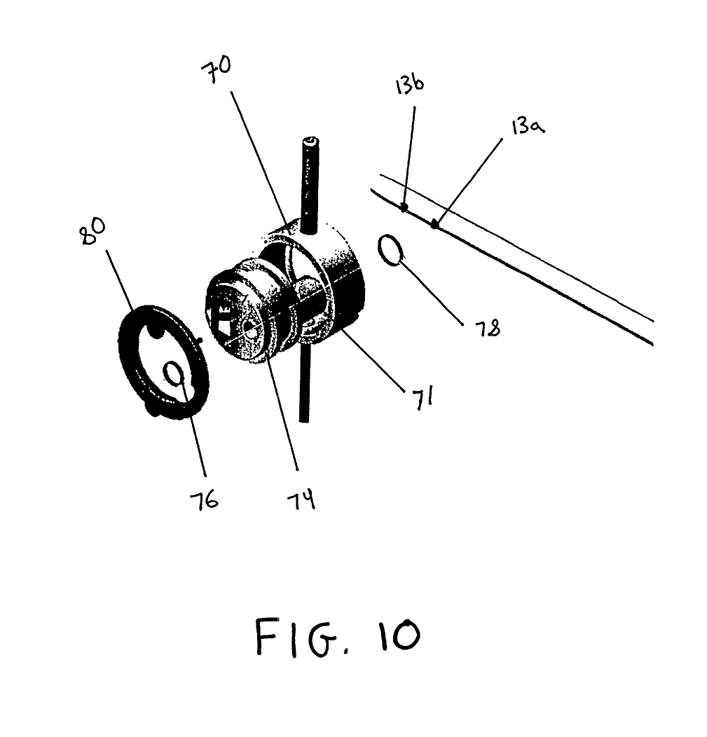

Referring now to FIG. 10, a washer 80 may secure and facilitate the clockwise and counter-clockwise rotation of an aiming cylinder support 74 within internal ring 70. Aiming cylinder support 74 may house an aiming cylinder 71 that is secured by an aiming cylinder locking screw 72, as shown in FIG. 8. A setting screw 61, also shown in FIG. 8, may secure drilling guide 95 in place inside aiming cylinder 71. Drilling guide 95 is typically furnished by the manufacturer of the intramedullary nail 12. It may be introduced into aiming cylinder 71 at the end of the aiming procedure. A drill may then be introduced through drilling guide 95 to drill a hole in the bone. Both the aiming cylinder support 74 and aiming cylinder 71 may be fabricated from a radio translucent material, or any other suitable surgical grade bio-compatible material.

According to an embodiment of the invention, aiming cylinder 71 may be surrounded on each end by concentric radio-opaque aiming rings. In particular, aiming cylinder 71 may be surrounded on a first end exactly and concentrically by an inner aiming ring 76. Inner aiming ring 76 may have a diameter which is slightly larger than the diameter of aiming cylinder 71 on it's first end. In addition, an outer aiming ring 78 (having a larger diameter than inner aiming ring 76) may be exactly and concentrically placed on a second end of aiming cylinder 71.

Successive adjustments of intermediate ring 66 and internal ring 70, as described above, may facilitate a final alignment between aiming cylinder 71 and a first distal hole, such as, for example, distal screw hole 13a. As the alignment is being finalized, a perfectly concentric superposition of inner aiming ring 76, outer aiming ring 78, and distal screw hole may have to be obtained for alignment to be correct.

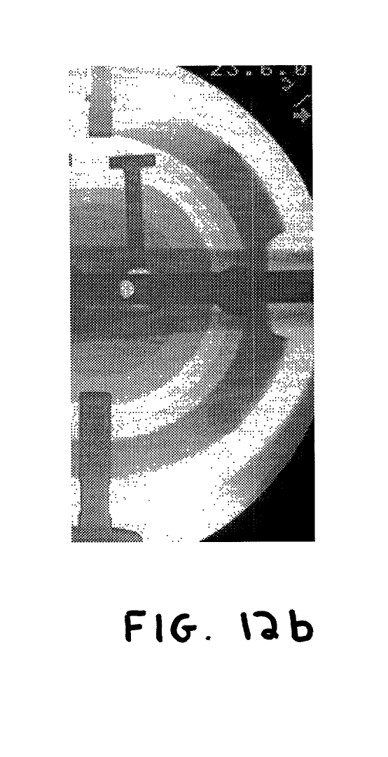

FIG. 12A is an illustration of a correct concentric alignment of aiming cylinder 71 with a screw hole in an implanted intramedullary nail. FIG. 12b, by contrast, illustrates an improper alignment of aiming cylinder 71 with a screw hole in an implanted intramedullary nail. To assist in the process of properly aligning aiming cylinder 71 with a screw hole, one or more reference points 69 for a neutral position may be placed on aiming guide 60, as shown in FIG. 8.

Aiming cylinder 71 may be eccentrically positioned (e.g., located elsewhere than at the geometric center) with regard to the center of rotation of internal ring 70. The distance from the center of aiming cylinder 71 to the center of rotation of internal ring 70 is approximately one-half the distance measured between the centers of the distal screw holes (13a, 13b) in intramedullary nail 12. The distance between the centers of distal screw holes (13a, 13b) is generally a known constant provided by the manufacturer of intramedullary nail 12.

Accordingly, once a first distal screw hole 13a has been accurately and finally aligned, the eccentric position of aiming cylinder 71 may enable a rapid-alignment of second distal screw hole 13b by making a 180 degree rotation of internal ring 70 about axis B.

Other embodiments, uses and advantages of the invention will be apparent to those skilled in the art from consideration of the specification and practice of the invention disclosed herein. The specification should be considered exemplary only, and the scope of the invention is accordingly intended to be limited only by the following claims.

Summary of reference characters

4-bone

5-fracture

6a-proximal bone segment

6b-distal bone segment

10-targeting system

12-intramedullary nail

13a, 13b-distal screw holes

14-nail handle

17a, 17b-distal locking fasteners

20-first fixation system

22-clamp support

24-clamp

25-first arm

26-adjustment mechanism

27-second arm

28-connection system

40-second fixation system

42-translation rod

44-locking screw

46-pressure spring

48-tubular clamp

49-plurality of notches

60-aiming guide

61-drilling guide setting screw

62-external ring

63a, 63b-translation adjustment screw

64a, 64b-translation adjustment square

65a, 65b-translation adjustment button

66-intermediate ring

67a, 67b-bearing

68a, 68b-fixing screw

69-reference points

70-internal ring

71-aiming cylinder

72-aiming cylinder locking screw

74-aiming cylinder support

76-inner aiming ring

78-outer aiming ring

95-drilling guide

What is claimed is:

1. A targeting system for locating screw holes in an intramedullary nail that has been implanted in a patient, the targeting system comprising:
a first fixation system adapted to be fixedly coupled to a surface;
a second fixation system adapted to be fixedly coupled to a handle of an intramedullary nail that has been implanted in a patient; and
an aiming guide disposed between and fixedly coupled to the first and second fixation systems, wherein adjustments to the first and second fixation systems enable an initial positioning of the aiming guide with a first screw hole in an implanted intramedullary nail, and adjustments to the aiming guide enable a final alignment of the aiming guide with the first screw hole.

2. The targeting system of claim 1, wherein the first fixation system further comprises a first arm that is linked to a clamp support at a first end, and releasably engaged at an opposite end to a second arm that is fixedly coupled to the aiming guide.

3. The targeting system of claim 2, wherein the clamp support houses a clamp adapted to be fixedly coupled to a surface that is integral with an operating table.

4. The targeting system of claim 2, wherein the first arm and second arm are releasably engaged via an adjustment mechanism.

5. The targeting system of claim 2, wherein the first end of the first arm is linked to the clamp support via a connection system.

6. The targeting system of claim 5, wherein the connection system comprises a ball and socket joint.

7. The targeting system of claim 2, wherein the second arm is linked to the aiming guide via a connection system.

8. The targeting system of claim 7, wherein the connection system comprises a ball and socket joint.

9. The targeting system of claim 1, wherein the second fixation system further comprises a tubular clamp adapted to be fixedly coupled to the handle of the implanted intramedullary nail.

10. The targeting system of claim 9, wherein the second fixation system further comprises a translation rod, the translation rod having a first end that is fixedly coupled to the aiming guide, and a second end that slidingly engages the tubular clamp.

11. The targeting system of claim 10, wherein a locking screw is disposed through the tubular clamp to control the rotation of the translation rod within the tubular clamp.

12. The targeting system of claim 10, wherein the translation rod further comprises a plurality of spaced notches at spaced intervals along its length that are adapted to correspond to scaled measurements of the length of the implanted intramedullary nail.

13. The targeting system of claim 12, wherein the tubular clamp houses a pressure spring that is biased to engage any of the plurality of spaced notches in the translation rod to control the translational movement of the translation rod through the tubular clamp.

14. The targeting system of claim 1, wherein the aiming guide further comprises an external ring, an intermediate ring, and an internal ring.

15. The targeting system of claim 14, wherein the intermediate ring is adapted to translate along a central horizontal axis of the external ring, and rotate about the central horizontal axis of the external ring.

16. The targeting system of claim 14, wherein the internal ring is adapted to translate along a central vertical axis of the intermediate ring, and rotate about the central vertical axis of the intermediate ring.

17. The targeting system of claim 14, wherein an aiming cylinder support is positioned within the internal ring by a washer, the aiming cylinder support further housing an aiming cylinder that is eccentrically positioned with regard to the center of rotation of the internal ring and secured via an aiming cylinder locking screw.

18. The targeting system of claim 17, wherein the aiming cylinder is eccentrically positioned such that the distance measured from the center of rotation of the internal ring to the center of the aiming cylinder is one-half the distance measured between the centers of the screw holes in the implanted intramedullary nail.

19. The targeting system of claim 17, wherein the aiming cylinder support and the aiming cylinder are fabricated from radio translucent material.

20. The targeting system of claim 17, wherein the aiming cylinder support further supports a drilling guide setting screw adapted to secure a drilling guide in position within the aiming cylinder.

21. The targeting system of claim 18, wherein the aiming cylinder is surrounded on either end by concentric radio-opaque aiming rings.

22. The targeting system of claim 21, wherein the concentric radio-opaque aiming rings comprise an inner aiming ring disposed on a first end of the aiming cylinder, and having a diameter that is slightly larger than the diameter of the first end of the aiming cylinder, and an outer aiming ring disposed on a second end of the aiming cylinder and having a diameter that is slightly larger than the diameter of the inner aiming ring.

23. The targeting system of claim 22, wherein the aiming guide is adapted for use with radioscopy equipment to enable a final alignment of the aiming guide with the first screw hole in the implanted intramedullary nail, the final alignment being achieved when a perfectly concentric alignment of the inner aiming ring, outer aiming ring, and first screw hole is realized.

24. The targeting system of claim 23, wherein, upon final alignment of the aiming guide with the first screw hole, alignment of a second screw hole in the implanted intramedullary nail is achieved by rotating the internal ring 180 degrees about the central vertical axis of the intermediate ring.

25. A method for manufacturing a targeting system for locating screw holes in an intramedullary nail that has been implanted in a patient, the method comprising the steps of:
(a) providing a first fixation system adapted to be fixedly coupled to a surface;
(b) providing a second fixation system adapted to be fixedly coupled to a handle of an intramedullary nail that has been implanted in a patient;
(c) providing an aiming guide to be disposed between and fixedly coupled to the first and second fixation systems, wherein adjustments to the first and second fixation systems enable an initial positioning of the aiming guide with a first screw hole in an implanted intramedullary nail, and adjustments to the aiming guide enable a final alignment of the aiming guide with the first screw hole; and
(d) assembling the first fixation system, second fixation system, and aiming guide to form a targeting system.

26. A method for locating screw holes in an intramedullary nail that has been implanted in a patient, the method comprising the steps of:
(a) using radioscopy equipment to align a first screw hole in an intramedullary nail that has been implanted in a patient;
(b) introducing a targeting system between the radioscopy equipment and the patient, the targeting system comprising a first fixation system adapted to be fixedly coupled to a surface, a second fixation system adapted to be fixedly coupled to a handle of the implanted intramedullary nail, and an aiming guide adapted to be disposed between and fixedly coupled to the first and second fixation systems;
(c) adjusting the first and second fixation systems to enable an initial positioning of the aiming guide with the first screw hole; and
(d) adjusting the aiming guide to enable a final alignment of the aiming guide with the first screw hole.

27. An aiming guide for locating screw holes in an intramedullary nail that has been implanted in a patient, the aiming guide comprising:
an external ring;
an intermediate ring;
and an internal ring supporting an aiming cylinder, wherein adjustments to the intermediate ring and internal ring enable the aiming cylinder to be precisely aligned with a first screw hole in an implanted intramedullary nail.

28. The aiming guide of claim 27, wherein the intermediate ring is adapted to translate along a central horizontal axis of the external ring, and rotate about the central horizontal axis of the external ring.

29. The aiming guide of claim 27, wherein the internal ring is adapted to translate along a central vertical axis of the intermediate ring, and rotate about the central vertical axis of the intermediate ring.

30. The aiming guide of claim 27, wherein the aiming cylinder is housed within an aiming cylinder support and secured via an aiming cylinder locking screw, the aiming cylinder support further being positioned within the internal ring by a washer.

31. The aiming guide of claim 27, wherein the aiming cylinder is eccentrically positioned with regard to the center of rotation of the internal ring.

32. The aiming guide of claim 31, wherein the aiming cylinder is eccentrically positioned such that the distance measured from the center of rotation of the internal ring to the center of the aiming cylinder is one-half the distance measured between the centers of two screw holes in an implanted intramedullary nail.

33. The aiming guide of claim 30, wherein the aiming cylinder support and the aiming cylinder are fabricated from radio translucent material.

34. The aiming guide of claim 30, wherein the aiming cylinder support further supports a drilling guide setting screw adapted to secure a drilling guide in position within the aiming cylinder.

35. The aiming guide of claim 32, wherein the aiming cylinder is surrounded on either end by concentric radio-opaque aiming rings.

36. The aiming guide of claim 35, wherein the concentric radio-opaque aiming rings comprise an inner aiming ring disposed on a first end of the aiming cylinder, and having a diameter that is slightly larger than the diameter of the first end of the aiming cylinder, and an outer aiming ring disposed on a second end of the aiming cylinder and having a diameter that is slightly larger than the diameter of the inner aiming ring.

37. The aiming guide of claim 36, wherein the aiming guide is adapted for use with radioscopy equipment to enable a final alignment of the aiming guide with the first screw hole in the implanted intramedullary nail, the final alignment being achieved when a perfectly concentric alignment of the inner aiming ring, outer aiming ring, and first screw hole is realized.

38. The aiming guide of claim 37, wherein, upon final alignment of the aiming guide with the first screw hole, alignment of a second screw hole in the implanted intramedullary nail is achieved by rotating the internal ring 180 degrees about the central vertical axis of the intermediate ring.

39. A method for manufacturing an aiming guide for locating screw holes in an intramedullary nail that has been implanted in a patient, the method comprising the steps of:
(a) providing an external ring;
(b) providing an intermediate ring; and
(c) providing an internal ring supporting an aiming cylinder, wherein adjustments to the intermediate ring and internal ring enable the aiming cylinder to be precisely aligned with a first screw hole in an implanted intramedullary nail.

40. A method for locating screw holes in an intramedullary nail that has been implanted in a patient, the method comprising the steps of:
(a) using radioscopy equipment to align a first screw hole in an intramedullary nail that has been implanted in a patient;
(b) introducing an aiming guide between the radioscopy equipment and the patient, the aiming guide comprising an external ring, an intermediate ring, and an internal ring supporting an aiming cylinder; and
(c) adjusting the positions of the intermediate ring and internal ring to enable the aiming cylinder to be precisely aligned with a first screw hole in an implanted intramedullary nail.
